(19) United States
(12) Patent Application Publication
(10) Pub. No.: US 2003/0138520 A1
(43) Pub. Date: Jul. 24, 2003

(54) Confectionery products containing active ingredients

(76) Inventors: David Alan Bell, York (GB); Emma Pickford, Epalinges (CH)

Correspondence Address: WINSTON & STRAWN, PATENT DEPARTMENT, 1400 L STREET, N.W., WASHINGTON, DC, 20005-3502, US

(21) Appl. No.: 10/328,913

(22) Filed: Dec. 20, 2002

Related U.S. Application Data
(63) Continuation of application No. PCT/EP01/06363, filed on Jun. 6, 2001.

(30) Foreign Application Priority Data
Jun. 30, 2000 (GB) 0016173.7

(51) Int. Cl. A23G 3/30

(52) U.S. Cl. 426/3

(57) ABSTRACT

A confectionery product, e.g., chocolate, containing one or more active ingredients therein. These active ingredients are incorporated in a plurality of carrier bodies that are dispersed within the body of the confectionery product.

### CROSS-REFERENCE TO RELATED APPLICATIONS

This application is a continuation of the US national stage designation of International application PCT/EP01/06363, filed Jun. 6, 2001 the entire content of which is expresssly incorported herein by reference thereto.

### FIELD OF THE INVENTION

The present invention relates to confectionery products containing active nutritive/functional ingredients, also known as dietary supplements.

### BACKGROUND OF THE INVENTION

Active nutritive or functional ingredients, which will hereinafter be referred to as “active ingredients”, include substances which are part of the daily diet such as vitamins and minerals, dietary supplements and herbal products, all of which are considered to be beneficial to the health of a person and may also give a feeling of well-being.

Chocolate confectionery products are known which contain active nutritive ingredients dispersed in minute particulate form or as dissolved solids throughout the product and are therefore invisible to the consumer and thus confer the element of functionality. It would be desirable if the active ingredients could be presented in the chocolate confectionery product in a form where their presence is obvious to the consumer both visibly and on eating the product. It is however undesirable to incorporate active ingredients in larger particulate form or in liquid form because larger particles can have an adverse effect on the texture or taste of the confectionery product. Also, functional ingredients are often required only in very small quantities. Some active ingredients are water-soluble and may not be compatible with chocolate.

Thus, there is a need for improvements in this area, and these are provided by the present invention.

### SUMMARY OF THE INVENTION

According to the present invention, there is provided a confectionery product comprising a basic confectionery component containing one or more active ingredients wherein the active ingredients are incorporated in a plurality of carrier bodies dispersed within the body of the confectionery product.

It has now been found that by dispersing active ingredients throughout a confectionery product in one or a plurality of carrier bodies, the incorporation of the active ingredient could be facilitated because it could be incorporated in much larger and visible particulate form or in liquid form. In addition, the carrier bodies could be made visible to the consumer on eating the confectionery product and may conveniently be of any desired shape, e.g. cubes, spheres, rectangles, triangles, polygons, stars, leaves, or irregular, random shape. These carrier bodies may also be in the form of strands running through the product or in layers.

The carrier bodies are preferably visually and/or texturally distinct from the surrounding confectionery product. Moreover, when the confectionery product contains more than one active ingredient, the formulation of the carrier bodies may, if necessary or desired, be adjusted to enable one active ingredient to be released quickly and another active ingredient to be released slowly. An additional advantage of concentrating functional ingredients into one or more discrete components of the product minimizes production plant contamination.

### DETAILED DESCRIPTION OF THE PREFERRED EMBODIMENTS

The basic confectionery component may be any confectionery material and may contain one or more additional confectionery materials different to the basic confectionery component.

Examples of confectionery material include chocolate, biscuit, wafer, nougat, praline, nuts, fruit, jelly, fondant cream, paste, caramel, chews, chewing gum, etc. For example, if the basic confectionery component is chocolate, it may contain additionally one or more additional confectionery materials different to chocolate, e.g., biscuit, wafer, nougat, praline, nuts, fruit, jelly, fondant cream, paste, caramel, etc.

When the basic confectionery component contains additional confectionery materials, these confectionery materials may be arranged in any number of dispositions, e.g. layers, clusters, stripes, etc.

When the basic confectionery component is chocolate, the chocolate may be, for instance, dark, milk or white chocolate, or it may be a fat containing confectionery material including chocolate substitutes and fat containing direct cocoa butter replacements, stearines, coconut oil, palm oil, butter or any mixture thereof; nut pastes such as peanut butter; praline; confectioner's coatings also known as compound or couvertures, used for covering ice cream or cakes usually comprising chocolate analogues with cocoa butter replaced by a cheaper non-tempering fat; or “Caramac” a product sold by Nestle comprising non-cocoa butter fats, sugar and milk.

The active ingredient may be any vitamin, enzyme, amino-acid supplement, protein, gum, carbohydrate, phytochemical, dextrose, lecithin, other trace nutrient, brain-stimulating substance, energy provider, a mineral, mineral salt, botanical extract, antioxidant, prebiotic, probiotic bacteria, fatty acid, oat beta glucan or other functional fiber, creatine, carnitine, bicarbonate, citrate, or any mixture thereof.

The mineral may be one or more of calcium, iron, selenium, zinc, magnesium, phosphorus, iodine, manganese, iron, boron or copper, molybdenum, potassium, chromium, vanadium or fluoride.

The phytochemical may be a polyphenol, procyanidin or a phenolic acid, catechin or epicatechin, isoflavone, terpene or other phytonutritive plant material.

The botanical extract may be selected from Guarana, Gingko Biloba, Kola nut, Goldenseal, Golo Kola, Schizandra, Elderberry, St. John's Wort, Valerian and Ephedra, beta-sitosterol, caffeine, cafestol, D-limonene, kabweol, nomilin, oltipraz, sulphoraphane, tangeretin, black tea, white tea, java tea, folic acid, garlic oil, fiber, green tea extract, lemon oil, mace, licorice, menthol, onion oil, orange oil, rosemary extract, milk thistle extract, Echinacea, Siberian ginseng or Panax ginseng, lemon balm, Kava Kava, matte, bilberry, soy, grapefruit, seaweed, hawthorn, lime blossom, sage, clove, basil, curcumin, taurine, wild oat herb, dandelion, gentian, aloe vera, hops, cinnamon, peppermint, grape, chamomile, fennel, marshmallow, ginger, slippery elm, cardamon, coriander, anise, thyme, rehmannia, eucalyptus, menthol, kava kava, schisandra, withania, cowslip, lycium, passion flower.

The antioxidant substance may be glutathione peroxidase, superoxide dismutase, catalase, co-enzyme Q10 or honey.

The prebiotic may contain fructose, galactose, mannose, soy or inulin.

The probiotic bacteria may be lactobacilli or bifidobacteria, lactococcus, streptococcus, leuconostoc, pediococcus or enterococcus.

The carrier bodies may be in the form of pellets or capsules, or they may be in the form of strands running through the product or they may be in the form of layers. The carrier bodies are advantageously visible and identifiable to the consumer on eating the product and may conveniently be of any desired shape, e.g., cubes, spheres, rectangles, triangles, polygons, stars, leaves, shaped like DNA such as NERDS®, or irregular/random shapes. They may all be of approximately the same size or may be of varying, random sizes. The carrier bodies are preferably visually or texturally distinct from the surrounding confectionery product and easily identifiable to the consumer on eating the product. If desired, a flavor and/or a color or reflective material may be incorporated in the carrier bodies, e.g., the flavor may be strawberry, raspberry, orange, mint, etc., and may be colored.

The carrier bodies could be made from any one of a number of possible materials which is selected to provide the desired visual, textural and flavor contrast to the confectionery product. Such materials could be jelly, pressed sugar, fudge, boiled sugar, biscuit, crystals of sugar, e.g., dextrose, or polyols with or without coating, starch-based, protein-based, e.g., gelatin, or any other edible item. One example of the carrier bodies comprises crystals of dextrose panned with dextrose shaped like DNA. When the carrier body is a capsule, it may be composed of an edible outer shell (e.g., gelatin) with a liquid filling inside.

The carrier bodies may have a volume of, for instance, up to 0.1 to over 10,000 cubic millimeters, preferably from 0.125 to 8000 cubic millimeters, and more preferably from 1 to 1000 cubic millimeters.

The carrier bodies may contain from up to 0.00000001 to 100% by weight of active ingredient and preferably from 0.000001 to 50% by weight of active ingredient. Some active ingredients have high functional activity at very low doses, such as vitamins and minerals (micronutrients), whereas others such as dextrose (macronutrients) are beneficial to the body in much higher amounts. Furthermore, plant extracts may only contain small amounts of active constituents within the bulk of the extract and may therefore need to be added in larger amounts to ensure sufficient effective quantities of the active parts.

The proportion of carrier bodies added to the confectionery product may be from 0.1% (or less) up to 99%, and preferably from 5% to 50% by weight of finished product.

The number of carrier bodies in the confectionery product may depend on the requirements. For example, there may be just one carrier body but more usually there will be a plurality, e.g., from 2 up to 1,000.

The confectionery product may be of any shape, but conveniently in the shape of a bar.

When the basic confectionery material is chocolate, the chocolate product may be prepared by conventional methods or by cold extrusion described in EP 0603467B. The carrier bodies containing the active ingredients may be added at a critical point in the process to ensure the structure of the carrier bodies remains intact and the ingredients remain biochemically viable e.g. by adding to tempered liquid chocolate just before depositing into molds.

### EXAMPLE

The following Example further illustrates the present invention. Percentages are given by weight.

A milk chocolate bar is prepared containing orange flavored and colored pectin jelly pieces to which have been added vitamin C (ascorbic acid). The jelly pieces are hemispheres of approximately 5 mm radius. The finished 50 g bar contains 15% of the RDA (recommended daily amount)of vitamin

The product is prepared as follows:

The jelly pieces are prepared as follows:

| | | |
|---|---|---|
| Low Methoxyl Pectin | 19 | g |
| Sugar, White | 50 | g |
| Sodium Citrate | 3.1 | g |
| Water, warm | 400 | g |

Pre-mix the dry ingredients.

Add slowly to the warm water and with continual stirring slowly bring it to the boil.

| | | |
|---|---|---|
| Sugar, White | 222 | g |
| Glucose Syrup, 42DE | 250 | g |

Add to part 1, return to the boil and cook to 80.0% SS.

| Flavor, Orange | 1.5 | g |
|---|---|---|
| Color, Orange | 1.0 | g |
| Citric Acid | 7.0 | g |
| Ascorbic Acid BP (Vitamin C) | 0.57 | g |
| (Note the quantity of ascorbic acid is based on | | |
| providing 15% of the RDA for Vitamin C in the | | |
| finished product - it does NOT allow for any | | |
| losses during the manufacturing process or during | | |
| storage) | | |
| Theor. Batch Yield | 632 | g |

Procedure:

Add and stir in the ingredients of part 3.

Deposit into starch impressions to form small hemispherical shaped pieces of approximately 5 mm radius.

When the jelly is set remove from starch.

Chocolate Bar

| | | |
|---|---|---|
| Milk chocolate | 40 g | 80.0% |
| Jelly pieces, above | 10 g | 20.0% |
| | 50 g | 100.0% |

Procedure:

Temper the chocolate and add the jelly pieces.

Mix to give an even distribution of jelly pieces in the chocolate

Deposit into bar mold and cool to set

The finished bar therefore provides 9 mg of ascorbic acid which is equivalent to 15% of the European RDA for Vitamin C of 60 mg (not allowing for any losses during processing or storage). The ascorbic acid is present in a plurality of orange colored carrier bodies easily identifiable to the consumer.

What is claimed is:

1. A confectionery product comprising a basic confectionery component containing one or more active ingredients incorporated in a plurality of carrier bodies dispersed within the body of the confectionery product.

2. The product of claim 1, wherein the basic confectionery component is chocolate, biscuit, wafer, nougat, praline, nuts, fruit, jelly, fondant cream, paste, caramel, chews or chewing gum.

3. The product of claim 1, wherein the basic confectionery component contains one or more additional confectionery materials that are different from the basic confectionery component.

4. The product of claim 1, wherein when the basic confectionery component contains additional confectionery materials arranged therein.

5. The product of claim 1, wherein the active ingredient is any vitamin, enzyme, amino-acid supplement, protein, gum, carbohydrate, phytochemical, dextrose, lecithin, other trace nutrient, brain-stimulating substance, energy provider, a mineral, mineral salt, botanical extract, antioxidant, prebiotic, probiotic bacteria, fatty acid, oat beta glucan or other functional fibre, creatine, carnitine, bicarbonate, citrate or any mixture thereof.

6. The product of claim 5, wherein the active ingredient is a mineral selected from the group consisting of calcium, iron, selenium, zinc, magnesium, phosphorus, iodine, manganese, iron, boron, copper, molybdenum, potassium, chromium, vanadium and fluoride.

7. The product of claim 5, wherein the active ingredient is a phytochemical selected from the group consisting of a polyphenol, procyanidin, phenolic acid, catechin, epicatechin, isoflavone, terpene and other phytonutritive plant materials.

8. The product of claim 5, wherein the active ingredient is a botanical extract selected from the group consisting of Guarana, Gingko Biloba, Kola nut, Goldenseal, Golo Kola, Schizandra, Elderberry, St. John's Wort, Valerian and Ephedra, beta-sitosterol, caffeine, cafestol, D-limonene, kabweol, nomilin, oltipraz, sulphoraphane, tangeretin, black tea, white tea, java tea, folic acid, garlic oil, fiber, green tea extract, lemon oil, mace, licorice, menthol, onion oil, orange oil, rosemary extract, milk thistle extract, Echinacea, Siberian ginseng or Panax ginseng, lemon balm, Kava Kava, matte, bilberry, soy, grapefruit, seaweed, hawthorn, lime blossom, sage, clove, basil, curcumin, taurine, wild oat herb, dandelion, gentian, aloe vera, hops, cinnamon, peppermint, grape, chamomile, fennel, marshmallow, ginger, slippery elm, cardamon, coriander, anise, thyme, rehmannia, eucalyptus, menthol, kava kava, schisandra, withania, cowslip, lycium, and passion flower.

9. The product of claim 5, wherein the active ingredient is an antioxidant selected from the group consisting of glutathione peroxidase, superoxide dismutase, catalase, co-enzyme Q10 and honey.

10. The product of claim 5, wherein the active ingredient is a prebiotic that contains fructose, galactose, mannose, soy or inulin.

11. The product of claim 5, wherein the active ingredient is a probiotic bacteria selected from the group consisting of lactobacillus, bifidobacterium, lactococcus, streptococcus, leuconostoc, pediococcus, and enterococcus.

12. The product of claim 1, wherein the carrier bodies are in the form of pellets or capsules, or they are in the form of strands or stripes running through the product or they are in the form of layers.

13. The product of claim 1, wherein the carrier bodies are visible and identifiable to the consumer when eating the product.

14. The product of claim 1, wherein the carrier bodies are shaped as cubes, spheres, rectangles, triangles, polygons, stars, leaves or irregular or random shapes.

15. The product of claim 1, wherein the carrier bodies are all of approximately the same size.

16. The product of claim 1, wherein the carrier bodies are of different sizes.

17. The product of claim 1, wherein the carrier bodies are visually or texturally distinct from the surrounding confectionery product.

18. The product of claim 1, further comprising a flavor, a color, or a reflective material incorporated in the carrier bodies.

19. The product of claim 1, wherein the carrier bodies comprise jelly, pressed sugar, fudge, boiled sugar, biscuit or another edible item.

20. The product of claim 11, wherein the carrier body is a capsule composed of an edible outer shell and containing therein a liquid containing the active ingredient.

21. The product of claim 11, wherein the carrier body is a capsule composed of an edible outer shell comprising gelatin.

22. The product of claim 1, wherein the carrier bodies have a volume of from 0.125 to 8000 cubic millimeters.

23. The product of claim 1, wherein the carrier bodies contain from 0.00000001 to 100% by weight of active ingredient.

24. The product of claim 1, wherein the carrier bodies contain from 0.000001 to 50% by weight of active ingredient.

25. The product of claim 1, wherein the carrier bodies are present in an amount of from 0.1% up to 99% by weight of the product.

26. The product of claim 1, wherein from 1 to 10,000 carrier bodies are present in the product.

27. The product of claim 1, wherein the confectionery product is in the shape of a bar.

28. A process of preparing a confectionery product wherein carrier bodies containing one or more active ingredients are added to a basic confectionery component in liquid form and solidifying the basic confectionery product containing the carrier bodies to ensure the structure of the carrier bodies remains intact and the ingredients remain biochemically viable.

29. The process of claim 28, wherein the basic confectionery component is chocolate and the carrier bodies containing one or more active ingredients are added to the tempered chocolate in liquid form, and cooling to set the liquid tempered chocolate.
